(19) United States
(12) Patent Application Publication
(10) Pub. No.: US 2020/0339945 A1
(43) Pub. Date: Oct. 29, 2020

(54) CELL CULTURES COMPRISING POLY(OXAZOLINE) STABILIZERS AND USE OF POLY(OXAZOLINES) FOR STABILIZING CELL CULTURES

(71) Applicant: Friedrich-Schiller-Universitaet Jena, Jena (DE)

(72) Inventors: Meike Nicole LEISKE, Jena (DE); Anja TRÄGER, Trebgast (DE); Ulrich Sigmar SCHUBERT, Jena (DE)

(21) Appl. No.: 16/616,727

(22) PCT Filed: May 28, 2018
(86) PCT No.: PCT/EP2018/000275

§ 371 Date: Nov. 26, 2019

(30) Foreign Application Priority Data
May 26, 2017 (DE) 10 2017 005 048.1

(51) Int. Cl. C12N 5/00; C08G 73/02

(52) U.S. Cl. CPC C12N 5/0031; C12N 5/005; C12M 27/20; C12N 2500/50; C08G 73/0233

(57) ABSTRACT

Cell cultures comprising poly(oxazoline) stabilizers and use of poly-(oxazolines for stabilizing cell cultures

Described are cell cultures containing in a cell culture medium one or more water-soluble poly(oxazolines).

The water-soluble poly(oxazoline) acts as a stabilizer for the cells and reduces the mechanical stress exerted by agitating the cell culture medium on the cells. This leads to an improved survival rate of the cells compared to the unstabilized state.

Cell cultures comprising poly(oxazoline) stabilizers and use of poly-(oxazolines for stabilizing cell cultures

The invention relates to the field of cultivation of cells in a cell culture medium and to the use of poly(oxazolines) for the stabilization of cell cultures.

Described are cell cultures containing in a cell culture medium one or more water-soluble poly(oxazolines). The water-soluble poly(oxazoline) acts as a stabilizer for the cells and reduces the mechanical stress exerted on the cells by moving the cell culture medium. This leads to an improved survival rate of the cells compared to the unstabilized state.

The cultivation of cells in cell culture media outside an organism is known. Cell cultures are widely used in research, development and production.

In the cultivation of cells, suspensions of cells or cells adhering to surfaces are allowed to grow in a cell culture medium.

The aim is to achieve the highest possible volume density of cells in the cell culture medium or on the surface. This promises an increased yield of cells and of desired metabolic products, but can also lead to problems. As a result of the high cell activity, it can lead for the cells to a difficult supply of vital agents, for example of nutrients or oxygen. This is tried to prevent, among other things, by strongly stirring the nutrient medium. This inevitably causes turbulence in the cell culture medium or near the surfaces, which in turn can mechanically strain the cells and thus damage them.

From the prior art, the addition of selected water-soluble polymers to cell culture media is already known and proven. Typical additives in this context are polyethyleneglycol polypropyleneglycol block copolymers, which are available e.g. as Kolliphor® P188 or as Pluronic® F68. By using these stabilizers, the strain on the cells can be reduced.

S. Lueck et al. and M. Platen et al. synthesized hydrogel-based “microbeads” from methacrylate monomers crosslinked with poly(oxazolines). These polymers serve as biodegradable hydrogel transport systems for stem cell cultures. In this case, water-insoluble matrices were constructed by means of polymers in order to cultivate the few stem cells gently and “species-appropriately”. The polymers are therefore not available in solution. This is fundamentally different from an approach in which polymers are given to the cell culture medium and these polymers are thus dissolved in this medium and are used for the cultivation of cells under stress. In doing so the cells are not firmly embedded in a matrix (Biomaterials, 2016, 79, 1-14, S. Lueck, R. Scubel, J. Rueb, D. Hahn, E. Mathieu, H. Zimmermann, D. Schwarnweber, C. Werner, S. Pautot, R. Jordan, Tailored and biodegradable poly(2-oxazoline) microbeads as 3D matrices for stem cell culture in regenerative therapies; Biomacromolecules, 2015, 16, 1516-1524, M. Platen, E. Mathieu, S. Lueck, R. Schubel, R. Jordan, S. Pautot, Poly(2-oxazoline)-based microgel for neuronal cell culture).

A. Dworak et al., A. Utrata-Wesolek et al. and P. I. Haris et al. functionalized surfaces by placing the reactive species of ring opening polymerization on an amine-functionalized surface in order to bind the polymers covalently to the surface while polymerization is terminated. These functionalized surfaces were then used in fibroblast cell cultures to reversibly pin them. Y. Chen et al. compared in this regard the properties of poly (2-methy-2-oxazoline-g-L-lysine) with poly (ethyleneglycol-g-lysine) and additionally address the serum stability of the polymers. They found that the poly(2-oxazoline) based system has a higher serum stability than the poly (ethyleneglycol) based system. A. Tait et al. used poly (2-methyl-2-oxazoline), poly (2-ethyl-2-oxazoline), poly (2-propyl-2-oxazoline) and poly (2-butyl-2-oxazoline) for a surface coating according to a similar principle and used the thus modified surfaces for the cultivation of tissues. Thus, in these approaches, immobilized polymers were used and both hydrophilic and hydrophobic polyoxazolines were used (J. Mater Sci. Mater. Med., 2014, 25, 1149-1163, A. Dworak, A. Utrata-Wesolek, N. Oleszko, W. Walach, B. Trzebicka, J. Aniol, A. L. Sieron, A. Klama-Baryla, M. Kawecki. Poly(2-substituted-2-oxazoline) surfaces for dermal fibroblasts adhesion and detachment; EP 2 574 664 A1, A. Utrata-Wesolek, W. Walach, N. Oleszko, A. Dworak, B. Trzebicka, A. Kowalczuk, J. Aniol, M. Lesiak, A. Sitkowska, A. L. Sieron, M. Kawecki, J. Glik, A. Klama-Baryla, M. Nowak, Method for preparation a thermosensitive coating on a substrate, the substrate with a thermosensitive coating and its application; Bio-Medical Materials and Engineering, 2004, 14, 419-425. P. I Haris, M. Dahm, J. Ruehe, B. Berchthold, D. Pruefer, O. Prucker, B.-J. Chang, A. Wallrath, H. Oelert, Ultrathin polymer monolayers for promotion of cell growth on bioprosthetic materials—Evolution of a new concept to improve long term performance of biologic heart vales; Biointerphases, 2014, 9, Y. Chen, B. Pidhatika, T. von Erlach, R. Konradi, M. Textor, H. Hall, T. Luhmann doi: 10.1116/14878461; Biomaterials, 2015, 61, 26-32, A. Tait, A. L. Fisher, T. Hartland, D. Smart, P. Glynne-Jones, M. Hill, E. J. Swindle, M. Grossel, D. E. Davies, Biocompatibility of poly(2-alkyl-2-oxazoline) brush surfaces for adherent lung cell lines).

R. Himmelreich, S. Werner and M. N. Leiske et al. used poly(2-oxazolines) to purify nucleic acids from biological samples. For this purpose, additional functional groups, such as amines, were introduced into these. These sources used functionalized poly(2-oxazolines) for purification of nucleic acids and not as a cell culture additive (EP 2 163 621 A1 corresponding to WO 2010/026167 A1, R. Himmelreich, S. Werner, Method and reagents for isolating and purifying nucleic acids from biological samples or from biochemical reactions by lysis, adhesion, washing and elution; Adv. Func. Mater., 2015, 25, 2458-2466, M. N. Leiske, M. Hartlieb, C. Paulenz, D. Pretzel, M. Hentschel, C. Englert, M. Gottschaldt, U. S. Schubert, Lab in a tube: Purification, amplification, and detection of DNA using poly(2-oxazoline) multilayers).

The polymer-based systems known in the prior art, which apply poly(2-oxazolines), used these immobilized in a cultivation vessel, but not as a medium additive in the cell culture. This is done to prevent the adhesion/growth of the cells on a surface. For this purpose, the surfaces are coated with biocompatible polymers that are immobilized on the surface.

Known polymer-based dissolved additives in cell cultures are the hydrophilic copolymers Pluronic® F68 or Kolliphor® P188 mentioned above.

It has now been surprisingly found that when selected poly(oxazolines) are used as stabilizers in cell culture media, the cultivation of cells at the same concentration results in an increased positive contribution to improve the survival rate of cells. The use of these selected poly(oxazolines) as stabilizers in cell culture media has not yet been described.

Partially hydrolyzed poly (2-ethyl-2-oxazolines) were used as additives to cultures of 3T3 fibroblasts, βTC3 pancreatic cells or P388.D1 macrophages (J. Mater Sci: Mater Med 26:157, p. 1-12, 2015, R. Shah, Z. Kronekova, A. Zahoranova, L. Roller, N. Saha, P. Saha, J. Kronek, In vitro study of partially hydrolyzed poly(2-ethyl-2-oxazolines) as materials for biomedical applications). In this work, the cytotoxicity of polyoxazoline-polyethyleneimine-copolymers was investigated, since linear polyethylene-imine is produced by hydrolysis from poly (2-ethyl-2-oxazoline). Poly(2-ethyl-2-oxazolines) were used as reference substances. To investigate cytotoxicity, the cells were cultured in the presence of foetal bovine serum (FBS) or of horse serum (HS), then mixed with the corresponding polymer and MTT solution and cultured for another 2 hours. After that, the cells were isolated and their absorbance at 595 nm was determined as a measure of their viability.

In a number of biotechnological applications, the use of serums in the cultivation of cells is not desired or possible. One reason for this, besides ethical aspects, is the naturally given batch fluctuation and the lack of reproducibility resulting from this as well as potential contamination and/or immunogenities of serums, which can lead to complications in patients. Furthermore, serums can contain impurities and transmit diseases. Therefore, in cell cultivation, to an increasing degree serum free culture media, better chemically defined media, are sought, in which components with more defined and reproducible properties are thus present.

The object of the present invention was the provision of cell cultures, which are characterized by excellent stability during and after cultivation and by an improved survival rate compared to the unstabilized state.

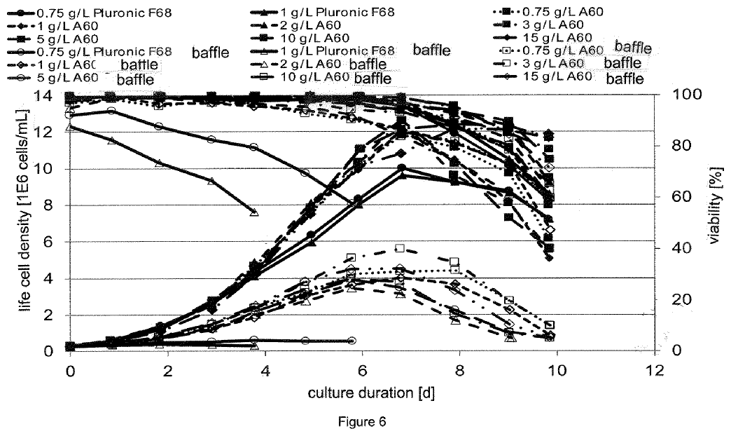

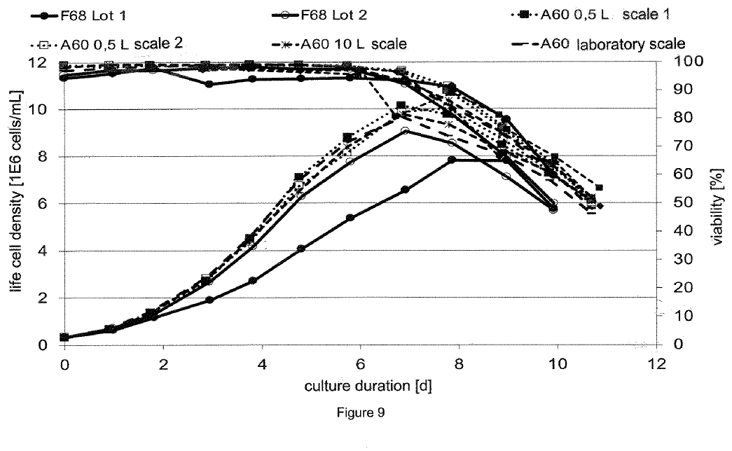

The technical problem is solved by the provision of cell cultures that contain one or more poly(oxazolines) dissolved in a cell culture medium. As mentioned above, the addition of selected water-soluble polymers, such as Pluronic® F68, is known from the prior art. As shown in FIG. 6 of the application, for example, the viability of a cell culture containing Pluronic® F68 decreased to less than 60% within 3 and 4 days, respectively. Furthermore, a so-called lot-to-lot variability was demonstrated for the additive Pluronic® F68 (Example 5). In contrast, the cell cultures of the present invention, which contain one or more poly (oxazolines) in a cell culture medium, showed significantly increased cell densities, increased viability and a longer culture duration even under high shear loads compared to the additive Pluronic® F68 (see FIG. 6 and Tables 1, 2 and 3). Furthermore, no significant difference between different tested poly(oxazoline) lots could be detected among each other in cell cultures (see, for example, FIG. 9 and Example 5).

Thus, the present invention relates to cell cultures containing one or more water-soluble poly (oxazolines) in the cell culture medium. Preferably, the present invention relates to cell cultures containing one or more water-soluble poly (oxazolines) in the cell culture medium, with the proviso that the cell culture medium substantially does not contain a serum. Consequently, the present invention preferably relates to cell cultures containing one or more water-soluble poly (oxazolines) in a cell culture medium, with the proviso that the cell culture medium used is substantially serum-free.

Preferred cell cultures according to the invention are characterized in that the cell cultures and in particular the cell culture medium are free of animal components. Furthermore, the invention relates to cell cultures, which are characterized by the fact that the cell culture medium used is protein-free. Particularly preferred in the context of the present invention are cell cultures, containing a cell culture medium, which is serum-free and/or protein-free.

Other preferred cell cultures according to the invention are those that contain cell culture media with only more defined chemical and/or biotechnological components. In other words, preferred cell cultures are those that are characterized by the fact that the cell culture medium is a chemically defined medium.

“Cells” are the smallest living units of organisms in the context of this description. These can be cells of single-celled organisms or of multi-cellular organisms, which can come from procaryotes or from eukaryotes. The cells can be microorganisms, individual cells or tissues. Cells can be of procariontic, plant or animal origin or can also come from fungi. Preferably, eukaryotic cells are used, in particular those that were originally isolated from tissues and can be permanently cultured, i.e. which are immortalized.

Under “cell culture” in the context of the present description combinations of cells and cell culture medium are designated, wherein the cells in the cell culture medium are cultivated outside the organism. Cell lines are used, i.e. cells of a tissue type that can reproduce in the course of cultivation. Both immortalized (immortal) cell lines and primary cells (primary culture) can be cultivated. Primary culture is usually a non-immortalized cell culture obtained directly from a tissue. “Cell culture medium” or “nutrient medium” is to be understood in the context of the present description as aqueous systems, which serve as a platform for the cultivation of cells. These systems contain all the substances needed for cell growth and viability.

In the context of this description, “cell culture media containing essentially no serum” are cell culture media which do not contain serum or only small amounts of up to 1 wt. % of serum, preferably less than 0.1 wt. %, relative to the cell culture medium.

The use of cell culture media in the pharmaceutical industry, for example for the manufacture of medicaments, such as active recombinant polypeptides, generally does not permit the use of any material of biological and/or animal origin due to safety and contamination problems. Therefore, the cell culture medium used according to the present invention is preferably a serum-free and/or protein-free medium.

In this description, the term “proteins” means proteins from more than 100 amino acids. Thus, the cell culture medium of the present invention may contain, for example, recombinant insulin (consisting of 51 amino acids).

The cell culture medium according to the present invention is also not supplemented with a hydrolysed protein source, such as soybean, wheat or rice peptone or yeast hydrolysate or the like.

Under “serum” in the context of the present description a blood serum or an immune serum is to be understood. Blood serum is the liquid portion of the blood that is obtained as a supernatant when a blood sample is centrifuged. This supernatant contains all substances naturally dissolved in the blood fluid, except for the coagulation factors consumed by clotting. The blood serum thus corresponds to the blood plasma minus the coagulation factors. Immunoserum is to be understood as a purification of specific antibodies derived from the blood serum of immunized mammals.

Serums in the context of this description usually mean serums from vertebrates, and in particular serums from calf, cow, cattle, horse or human.

“Cell culture media substantially free of animal components” means, in the context of the present description, cell culture media which have no or only small amounts of up to 1 wt. %, preferably less than 0.1 wt. %, relative to the cell culture medium of animal components. Typical animal components that are avoided in the cell cultures and cell culture media of the invention are serum and serum derived proteins, such as albumin, transferrin or other growth factors as well as recombinant forms thereof or protein from plant or yeast hydrolysates or ultrafiltrated forms thereof.

In the context of this description, “chemically defined cell culture media” means cell culture media which, in addition to water, additionally contain chemically and/or biotechnologically produced components, and in particular cell culture media consisting, in addition to water, exclusively of chemically and/or biotechnologically produced components. Typically, a “chemically defined cell culture medium” (also called “chemically defined medium”) is a term understood by the skilled person in the field of cell culture and cell culture media and is known to the skilled person. Consequently, the term “chemically defined cell culture medium” refers to a nutrient solution in which cells are contained and cultured and which generally provide at least one or more components from the following: an energy source (usually in the form of a carbohydrate such as glucose); all essential amino acids and generally the twenty basic amino acids, acids, plus cysteine; vitamins and/or other organic compounds typically needed in low concentrations; lipids or free fatty acids, e.g. linoleic acid; inorganic compounds or naturally occurring elements, which are typically contained in very low concentrations, usually in the micromolar area in the cell culture medium. Cell culture media can also be supplemented by a variety of optional components, such as salts, e.g. calcium, magnesium and phosphate, and buffers, e.g. HEPES; nucleosides and bases, e.g. adenosine, thymidine, hypoxanthine; antibiotics, e.g. gentamycin.

The term “chemically defined cell culture medium” thus stands for a completely chemically defined medium which does not contain additives from animal sources, such as tissue hydrolysates, e.g. fetal bovine serum or the like. Furthermore, proteins, in particular growth factors such as transferrin or recombinant forms thereof, are also not added to the cell culture according to the present invention.

Commercially available serum and/or protein-free cell culture media can be used in the present invention and include, for example, the media offered by Xell AG (Bielefeld) HEK TF (order no. 861), HEK GM (order no. 851), HEK FS (order no. 871), BHK medium (order no. 910), BHK FS (order no. 915), MDXK medium (order no. 1010), HYB GM (order no. 890), HYB FS (order no. 895), TCX6D medium (order no. 1070), TCX10D medium (order no. 1100), TCX7D (order no. 1080), CHO TF (order no. 886).

In the context of this description, “bioreactors” or “fermenters” are to be understood as vessels for the cultivation of cells in which the cells are in contact with a cell culture medium and can be cultivated under permanent movement to high cell densities. In doing so, the cells may be suspended in the cell culture medium or grow adherend on surfaces that are in contact with the cell culture medium. The purpose of cultivation in a bioreactor may be the production of cells or metabolites. The latter can be used, for example, as active ingredients in the pharmaceutical industry or as basic chemicals in the chemical industry. In bioreactors, several factors are usually controlled and/or monitored that influence the growth of cells. Examples are the composition of the nutrient medium, the oxygen supply, the temperature, the pH and the sterility. Different reactor variants can be used in different versions. Examples of this are stirrer tank reactors, e.g. those made of metal, which can have a volume of a few to thousands of liters and which are filled with nutrient solution. Other variants can also be used, such as fixed bed reactors, photobioreactors or fluidized bed reactors.

In the context of the present description “polymers” mean organic compounds characterized by repetition of certain units (monomer units or repeat units). Polymers may consist of one type or of several types of different repeat units. Polymers are produced by the chemical reaction of monomers under the formation of covalent bonds (polymerization) and form the so-called polymer backbone by connecting the polymerized units. This can have side chains where functional groups can be located. Homopolymers consist of only one monomer unit. Copolymers, on the other hand, consist of at least two different monomer units, which can be arranged statistically, as gradient, alternating or as block.

In the context of this description, “surfactants” are water-soluble substances or mixtures of substances used to stabilize cell cultures. They are usually added to the aqueous phase in the cultivation of cells and serve primarily to minimize the effects of shear forces on the cells and thus increase the viability of the cells.

“Water-soluble poly(oxazolines)” means, in the context of the present description, polyoxazolines, which dissolve to at least 10 g/L in water at 25° C.

The cells used according to the invention can be produced and cultured according to standard methods.

For example, primary cultures can be created from different tissues, for example from tissues of individual organs, such as skin, heart, kidney or liver, or from tumor tissues. The tissue cells can be individualized by known methods, e.g. by treatment with a protease, whereby the proteins that maintain the cell association are catabolized. It may also be appropriate to specifically stimulate some cell types for division by adding growth factors or, in the case of poorly growing cell types, to use feeding cells, basal lamina like matrices or recombinant components of the extracellular matrix. The cells used according to the invention can also be genetically modified by introducing a plasmid as a vector.

The cells used according to the invention may have a limited lifespan or they are immortal cell lines with the ability to divide infinitely. These may have been generated by random mutation, e.g. in tumor cells, or by targeted modification, for example by the artificial expression of the telomerase gene.

The cells used according to the invention may be adhesively (on surfaces) growing cells, such as fibroblasts, endothelium cells or cartilage cells, or they may be supension cells that grow freely floating in the nutrient medium, such as e.g. lymphocytes.

Preferably, cells are used that are suspended in the cell culture medium.

Particularly preferred, suspension-adapted cells are used. These are cells, preferably eukaryotic cells, which originally grow and are cultivated adherent, but can go into suspension by changing the ingredients of the medium and cultivation. This allows higher cell culture densities to be achieved.

Culture conditions and cell culture media are selected depending on the individual cultured cells. The different cell types prefer different culture media, which are specifically compiled. For example, different pH values are set and the individual culture media can contain different amino acids and/or other nutrients in different concentrations.

The cell cultures of the invention are used especially in the field of biotechnology. This can be the production of (recombinant) proteins, of virus and/or virus particle production, investigation of metabolism, division and other cellular processes. Furthermore, the cell cultures of the invention can be used as test systems, for example in the study of the effect of substances on cell properties, such as signal transduction or toxicity. The cell cultures of the invention can also be used for the production of biotechnical products. For example, for the preparation of chemical compounds, such as raw material chemicals or as active ingredients for pharmaceuticals, for example of monoclonal antibodies, proteins or vaccines. The cell cultures of the invention can also be used in plant breeding, for example in plant tissue culture, in which complete plants can be produced from cell cultures.

Preferably, the following cell lines are used in the cell cultures of the invention:

293-T: Species of origin human, tissue of origin kidney, morphology epithelium

A431: Species of origin human, tissue of origin skin, morphology epithelium

A549: Species of origin human, tissue of origin adenocarcinoma of the lungs, morphology epithelium

BCP1: Species of origin humans, tissue of origin blood, morphology lymphocyte

bEnd.3: Species of origin mouse, tissue of origin brain/cerebral cortex, morphology endothelium

BHK-21: Species of origin hamster, tissue of origin kidney (embryonic), morphology fibroblast

BxPC-3: Species of origin human; tissue of origin pancreas adenocarcinoma, morphology epithelium

BY-2: Species of origin tobacco, tissue of origin At the seedling induced callus

CHO: Species of origin hamster, tissue of origin ovaries, morphology epithelium

CMT: Species of origin dog, tissue of origin mammary gland, morphology epithelium

COS-1: Species of origin monkey, tissue of origin kidney, morphology fibroblast

COS-7: Species of origin monkey, tissue of origin kidney, morphology fibroblast

CV-1: Species of origin monkey, tissue of origin kidney, morphology fibroblast

EPC: species of origin fish, tissue of origin skin, morphology epithelium

HDMEC-T: Species of origin human, tissue of origin foreskin, morphology endothelium

HEK or HEK 293: species of origin human, tissue of origin kidney (embryonic), morphology epithelium

HeLa: Species of origin human, tissue of origin cervical carcinoma, morphology epithelium

HepG2: Species of origin human, tissue of origin liver cell carcinoma, morphology epithelium

HL-60: Species of origin human, tissue of origin promyeloblasts, morphology blood cells

HMEC-1: Species of origin human, tissue of origin foreskin, morphology endothelial

HUVEC-T: Species of origin human, tissue of origin umbilical cord vein, morphology endothelium

HT-1080: Species of origin human, tissue of origin fibrosarcoma, morphology connective tissue cells

Jurkat: Species of origin human, tissue of origin T-cell leukemia, morphology blood cells

K562: Species of origin human, tissue of origin leukemia, morphology myeloid blood cells

LNCaP: Species of origin human, tissue of origin prostate, morphology epithelium

MCF-7: Species of origin human, tissue of origin breast adenocarcinoma, morphology epithelium

MCF-10A: Species of origin human, tissue of origin mammary gland, morphology epithelium

MDCK II: Species of origin dog, tissue of origin kidney, morphology epithelium

MDT-1A: Species of origin mouse, morphology epithelium

MyEnd: Species of origin mouse, morphology endothelium

Neuro-2A: Species of origin mouse, tissue of origin brain, morphology neuroblast

NIH-3T3-T: Species of origin mouse, tissue of origin embryo, morphology fibroblast

NTERA-2 cl.D1: Species of origin human, tissue of origin testicles lung metastasis, morphology epithelium

P19: Species of origin mouse, tissue of origin embryonic carcinoma, morphology epithelium

PANC-1: Species of origin human, tissue of origin pancreas adenocarcinoma, morphology epithelium

Peer: Species of origin human, tissue of origin T-cell leukemia

RTL-W1-T: Species of origin rainbow trout, morphology fibroblast

Sf-9: Species of origin moth, tissue of origin ovar

Saos-2: Species of origin human, tissue of origin osteosarcoma, morphology epithelium

T2: Species of origin human, morphology T-cell leukemia

T84: Species of origin human, tissue of origin colorectal carcinoma lung metastasis, morphology epithelium

U-937: Species of origin human, tissue of origin Burkitt lymphoma, morphology monocytic.

Other cells preferably used in the cell cultures of the invention are those that are not fibroblasts, pancreatic cells or macrophages. In the cell cultures of the invention particularly preferably used cells are those that are no 3T3 fibroblasts, the βT3 pancreatic cells and the murine P388.D1 monocytes/macrophages.

Other cells preferably used in the cell cultures of the invention are hybridoma cells. These are known to be drug-producing cells that have been fused with cancer cells (immortalized cells), creating immortal hybrids.

Other cells preferably used in the cell cultures of the invention are stem cells. These are known to be body cells that can differentiate into different cell types or tissues.

Particularly preferred is the use of the cell lines CHO and HEK, preferably HEK 293.

The cell cultures of the invention contain one or more water-soluble poly (oxazolines). The amount of poly (oxazoline), based on the total amount of the cell culture according to the invention, is usually 0.01 to 15 wt. %, 0.1 to 15 wt. %, preferably 0.05 to 10 wt. %, more preferred 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 11, 12, 13, 14 to 15 wt. %, most preferably 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9 to 10 wt. %, and very most preferably 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9 to 10 wt %.

Poly(oxazolines) are known compounds. These are usually prepared by cationic ring opening polymerization of oxazolines, preferably of 2-oxazolines, in solution and in the presence of an initiator. Examples of initiators are electrophiles, such as salts or esters of aromatic sulfonic acids or carboxylic acids or salts or esters of aliphatic sulfonic acids or carboxylic acids or aromatic halogen compounds. Multi-functional electrophiles can also be used as initiators. In addition to linear poly(oxazolines), branched or star-shaped molecules can also be formed. Examples of preferred initiators are esters of arylsulfonic acids, such as methyl tosylate, esters of alkanesulfonic acids, such as trifluoromethane sulfonic acid, or mono- or dibromobenzene. The polymerization is usually carried out in a polar aprotic solvent, for example in acetonitrile.

As oxazolines for the preparation of the poly(oxazolines) used according to the invention, 2-oxazolines (4,5-dihydrooxazoles) with a C═N double bond between the carbon atom 2 and the nitrogen atom are used. These may be substituted at the 2-, 4- and/or 5-carbon atom and/or at the 3-nitrogen atom, preferably at the 2-carbon atom and/or at the 3-nitrogen atom.

Preferably, 2-oxazolines are used, which contain a substituent at 2 position. Examples of such substituents are methyl or ethyl.

Besides the 2-oxazolines, in the preparation of the water-soluble poly-(oxazolines) used according to the invention, still small amounts of further monomers copolymerizable with 2-oxazolines can be used.

The water-soluble poly (oxazolines) used according to the invention usually contain at least 80 wt. %, in particular at least 90 wt. % and most preferably at least 95 wt. %, based on their total mass, of repeat structural units of formula I and/or formula II

(I), —NR¹—CR³H—CR⁴H—CR⁵H— (II),

in which

R¹ means a residue of formula —CO—R²,

R³, R⁴ and R⁵ independently of one another mean hydrogen, methyl, ethyl, propyl or butyl,

R² is selected from the group consisting of hydrogen, methyl, ethyl, —CₘH₂ₘ—X or —(CₙH₂ₙ—O)ₒ-(CₚH₂ₚ-O)_{q}—R⁶,

R⁶ is hydrogen or C₁-C₆-alkyl, preferably methyl or very preferred hydrogen,

m is an integer from 1 to 6,

X is selected from the group consisting of hydroxyl, alkoxy, amino, N-alkylamino,

N,N-dialkylamino, carboxyl, carboxylic ester, sulfonyl, sulfonic acid ester or carbamate,

n and p independently of one another are integers from 2 to 4, wherein n is unlike p,

n is preferably 2 and p is preferably 3, and

o and q independently of one another are integers from 0 to 60, preferably from 1 to 20 and very preferred from 2 to 10, wherein at least one of o or q is unlike 0.

Preferred water-soluble poly(oxazolines) used according to the invention are those in which R² is hydrogen, methyl or ethyl and R³ to R⁵ mean hydrogen or in which R² is hydrogen, methyl or ethyl and two of the residues R³ to R⁵ are hydrogen and one of the residues R³ to R⁵ is methyl or ethyl.

The molar mass of the poly(oxazolines) used according to the invention is usually 2,500 to 500,000 g/mol, in particular 5,000 to 50,000 g/mol. The molar mass is determined for the purposes of this description by ¹H-NMR analysis. An indirect determination of the molar mass is also possible via end group determination and of the degree of polymerization and the molecular weight of the monomer by comparing the integrals of the protons with each other and thus determining the number of repeat units.

Particularly preferred cell cultures according to the invention contain a water-soluble poly(oxazoline), which possesses at least 90 wt. %, in particular at least 95 wt. %, based on its total mass, of repeat structural units of formula I, wherein R² means methyl or ethyl.

Further preferred cell cultures according to the invention contain in addition to the cells, the cell culture medium, the water-soluble poly(oxazoline) one or more active ingredients, in particular one or more pharmaceutical active ingredients.

The cultivation of the cell cultures according to the invention can be carried out in a known manner. For that bioreactors are used. These are filled with nutrient media and cells. Cultivation can be performed batchwise or in a continuous manner. The content of the bioreactor is agitated, preferably by stirring, wherein beneficial baffles are used for the generation of turbulence. The agitation of the reactor content can be carried out by known stirring devices and/or by injection of liquids.

The invention also relates to methods for protein production, virus and/or virus particle production, investigation of metabolism, division and/or further cellular processes, comprising the cultivation of the inventive cell cultures described herein.

The methods described herein also preferably contain the following measures:
(i) filling a bioreactor with cells and a cell culture medium described herein, preferably a serum-free and/or a protein-free cell culture medium, wherein the cell culture medium contains one or more water-soluble poly(oxazolines) described herein; and
(ii) agitating the contents of the bioreactor.

The temperature during cultivation and the composition of the nutrient medium are selected according to the needs of the cells to be cultivated.

Preferably, the cultivation of the cells takes place at temperatures between 31 and 39° C., especially at temperatures between 36 and 37° C. The cultivation of the cell cultures according to the invention can be carried out anaerobic or in particular aerobic. Typically, in addition to nitrogen, oxygen and noble gases, the atmosphere still contains CO₂, for example in amounts of 0.01 to 10 wt. %, especially in quantities of 0.01 to 5 wt. %, based on the mass of the atmosphere. Preferably, the atmosphere in the bioreactor consists of air.

The duration of the cultivation of the cells can be chosen in a wide range and can be performed batchwise or continuously with and without feeding nutrients. Typical cultivation periods for the batchwise procedures are 5 hours to 30 days, preferably 5 hours to 21 days and in particular 10 hours to 15 days. Typical cultivation periods for continuous procedures are 10 days to 180 days, preferably 10 days to 60 days and in particular 15 days to 35 days.

Depending on the rate of division and density of the cells, cell associations can be dissolved every few days and distributed to new vessels (also called “passage”). The number of passages indicates the frequency with which the cells have already been passaged. In adherent cells in continuous culture, the cells are preferably isolated regularly to avoid confluence and the associated cell contact inhibition.

The poly(oxazolines) used according to the invention can be added to the cell culture medium during cultivation and/or during a modification of the cell culture, such as a transfection.

After cultivation of the cell culture according to the invention in the bioreactor, this is preferably processed. The cells and/or the produced active agents are separated from the other ingredients. This can be performed by standard methods, for example by filtration or centrifuging.

The invention also relates to the use of the water-soluble poly(oxazolines) described herein for the stabilization of cell cultures. Preferably, as described herein above, the poly(oxazolines) are introduced into a cell culture medium described herein.

The following examples illustrate the invention without limiting it.

### Example 1A

### Manufacture of poly(2-oxazolines) (POₓ) in the Microwave

The synthesis of poly(2-oxazolines) has already been described in the literature (see e.g. F. Wiesbrock et al. Macromolecular Rapid Communications 2004, 25, 1895-1899). The procedure is therefore described as an example for poly (2-ethyl-2-oxazoline) with a degree of polymerization (DP) of 61 (P(EtOₓ)₆₁).

In a microwave reaction vessel, 2-ethyl-2-oxazoline (6.06 mL, 60.0 mmol), methyltosylate (0.15 mL, 0.1 mmol) and acetonitrile (8.79 mL) were mixed under inert conditions. The reaction vessel was then heated in a synthesis microwave for 14 min to 140° C. Subsequently, the reaction was terminated by the addition of 0.5 mL of deionized water and stirred overnight at room temperature. The resulting solution was purified by diluting with dichloromethane and then by precipitation in an excess of ice-cold diethyl ether. The precipitated polymer was then filtered and dissolved in dichloromethane. The solvent was then removed in a rotary evaporator and the polymer was dried in the high vacuum until completely solvent-free. The final product was available as a crystalline white solid.

¹H-NMR (CDCl₃, 300 MHz): δ=4.34 (0.1H, s, backbone-OH), 3.44 (4.0H, s, backbone), 3.02 (0.3H, s, CH₃-backbone), 2.4 (1.7H, m, CH₂ (EtOₓ)), 1.11 (2.5H, s, CH₃ (EtOₓ)) ppm.

SEC (eluent: DMAc¹), 0.21% LiCl, PS²)-Standard): Mₙ=11.200 g mol⁻¹, M_{w}=12.200 g mol⁻¹, D=1.09.

¹⁾ DMA_{c}=dimethyl acetamide

²⁾ PS=polystyrene

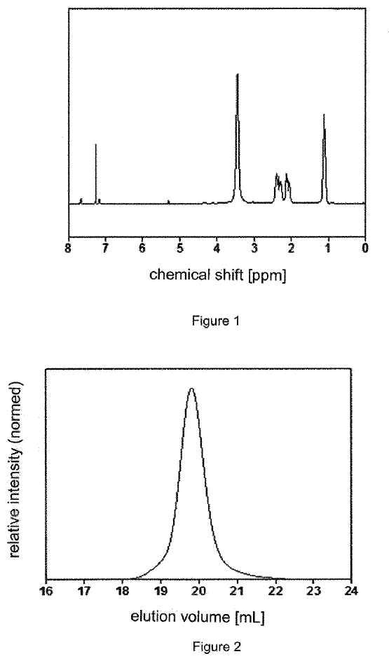

FIG. 1 shows a ¹H-NMR (300 MHz, CDCl₃) of the purified P(EtOₓ)₆₁.

FIG. 2 shows an SEC elugramme (DMA_{c}, 0.21% LiCl, PS calibration) of the purified P(EtOₓ)₆₁.

### EXAMPLE 1B

### Manufacture of POₓ in the Reactor

In the reactor, 2-ethyl-2-oxazoline (4.04 L, 40.0 mol), methyltosylate (100 mL, 0.67 mol) and acetonitrile (5.86 L) were mixed under inert conditions. The reactor was then heated under reflux for 6 hrs. Reaction progress was monitored by taking samples at regular intervals. Subsequently, the reaction was terminated by the addition of 270 mL of deionized water and stirred overnight at room temperature. The resulting solution was cleaned in five portions. For this purpose, the solvent was removed in the rotary evaporator. Subsequently, the polymer was dissolved in 4 L dichloromethane. Then the organic phase was washed with 2 L of a saturated sodium bicarbonate solution and then twice with 2 L of a saturated sodium chloride solution. The organic phase was dried over sodium sulfate and the solvent was evaporated in the rotary evaporator. Then the polymer was dried in the high vacuum until completely solvent-free. The final product was available as a crystalline white solid.

### EXAMPLE 2

### Cell Growth in a Baffled Flask

Suspension-adapted HEK-F cells were cultured by means of dynamic cell culture in serum free medium (deficiency variant of the HEK TF medium (serum and protein free, without Pluronic® F68) (order no. 861 (Xell AG, Bielefeld)) for at least 5 days without the addition of new medium. In a baffled flask (250 mL), the cells with a cell density of 0.3×10⁶ cells per mL were applied in 30-40 mL minimal medium with correspondingly added surfactant (750 mg L⁻¹). Cultivation took place in the incubator under shaking at 128 rpm, 37° C. and 5% CO₂. Every day, cell density and cell viability were determined by counting using trypan blue. A cell viability below 60% is considered as an abort criterion.

TABLE 1
Cell concentrations of living cells expressed in cells per millilitre

| day | Pluronic ® F68 | P(EtOx)₆₁ | P(MeOx)₅₇ |
|---|---|---|---|
| 0 | 300000 | 300000 | 300000 |
| 1 | 980000 | 720000 | 1400000 |
| 2 | 1644000 | 1816000 | 2012000 |
| 3 | 3045000 | 3308000 | 3044000 |
| 4 | 3728000 | 4304000 | 4904000 |
| 5 | 5296000 | 6152000 | 6040000 |
| 6 | 6030000 | 6960000 | 6488000 |
| 7 | 5670000 | 7288000 | 6752000 |
| 8 | 8020000 | 7640000 | 7820000 |

TABLE 2
Cell survival rate, expressed in %

| day | Pluronic ® F68 | P(EtOx)₆₁^{a)} | P(MeOx)₅₇^{b)} |
|---|---|---|---|
| 0 | 100 | 100 | 100 |
| 1 | 96.1 | 97.3 | 93.3 |
| 2 | 97.2 | 96.8 | 97.7 |
| 3 | 95.4 | 98.2 | 98.0 |
| 4 | 96.5 | 98.3 | 95.8 |
| 5 | 96.2 | 95.1 | 94.0 |
| 6 | 93.6 | 95.1 | 97.7 |
| 7 | 89.4 | 95.6 | 96.8 |
| 8 | 92.7 | 90.4 | 93.3 |

^{a)}poly(2-ethyl-2-oxazoline) with a polymerization degree (DP) of 61
^{b)}poly(2-methyl-2-oxazoline) with a polymerisation degree (DP) of 57

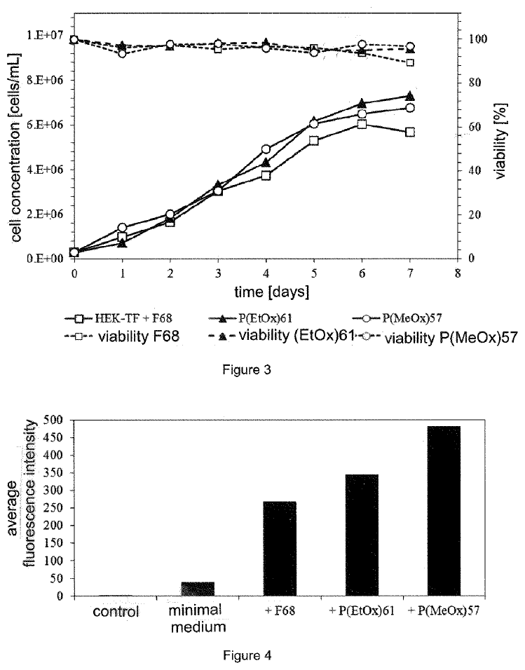

FIG. 3 shows the cell concentrations and viability of HEK F cells when cultivated with different surfactants.

### EXAMPLE 3

### Transfection of Suspension Cells

HEK-F cells were pre-cultivated in the corresponding minimal medium deficiency variant of the HEK TF medium (serum and protein free, without Pluronic® F68) (order no. 861 (Xell AG, Bielefeld)) with surfactant (750 mg L⁻¹). On the day of transfection, the cells were centrifuged and the cell density was adjusted with fresh medium to 3×10⁶ cells mL⁻¹. Transfection at N/P 20 (amine to phosphate ratio) was carried out in a 2 mL preparation as follows: First addition of 15 μg mL⁻¹ pDNA (EGFP reporter gene) to the cell suspension followed by pivoting this, then adding polyethyleneimine (PEI, 1 mg mL⁻¹) and repeated pivoting of the culture. The preparation was incubated for 4 h under shaking in the incubator at 37° C., 5% CO₂. After this incubation period, the cells were transferred to 6-well plates and diluted with the same volume of fresh medium and incubated for a further 48 h. Transfection efficiency was determined by flow cytometry.

Table 3 shows the results of transfection experiments of HEK-F cells in minimal medium

| | incubation | cell | transfected | average |
|---|---|---|---|---|
| | time | viability | cells | intensity of |
| | [hours] | [%] | [%] | fluorescence |
| negative control | 48 | 98.4 | 0.7 | 0.775 |
| minimal medium | 48 | 95.7 | 11.7 | 37.700 |
| Pluoronic ® F68 | 48 | 96.6 | 49.0 | 267.000 |
| P(EtOₓ)₆₁ | 48 | 96.3 | 60.3 | 344.000 |
| P(MeOₓ)₅₇ | 48 | 97.7 | 74.8 | 482.000 |

FIG. 4 shows the average fluorescence intensity of the transfected HEK F cells.

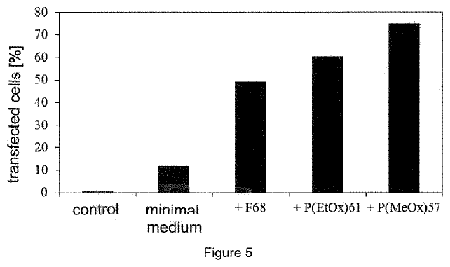

FIG. 5 shows an overview of the number of transfected cells, expressed in %, when using different culture media.

### EXAMPLE 4

### Variation of the Concentration Range of POₓ

In order to be able to estimate the concentration range in which POₓ can be used as a surfactant in cell culture processes, additional cultivations were carried out. HEK F cells were cultivated for this purpose in a deficiency variant of the HEK TF medium (serum and protein free, without Pluronic® F68) (order no. 861 (Xell AG, Bielefeld)) with the addition of the current standard surfactant Pluronic® F68 or P(EtOₓ). For Pluronic® F68 concentrations of 0.75 g/L and 1 g/L (most commonly used concentrations) were used and P(EtOₓ) was tested in a larger concentration range from 0.75 g/L to 15 g/L. In order to investigate also the protective property against increased shear forces, this test was carried out both in shaking flasks without baffle and with baffle. The results are shown in the lower graphs in FIGS. 6 to 8.

FIGS. 6 to 13 refer to P(EtOₓ) as A60.

FIG. 6 provides an overview of the growth curves and the viability course (dashed lines) of the HEK F cells in HEK TF medium. Different concentrations Pluronic® F68 (0.75 g/L and 1 g/L) and P(EtOₓ) (0.75-15 g/L) were used as surfactants. A shaking flask with baffle (baffled, dotted lines) and a shaking flask without baffle (plain, solid lines) were used to expose the cells to different shear forces.

FIG. 6 shows an overall overview of the concentration testing of Pluronic® F68 against P(EtOₓ). In general, the maximum cell densities in the shaking flasks without baffles were significantly higher than those in shake flasks with baffles (9.6-12.9-10⁶ cells/mL compared to 0.2-5.6×10⁶ cells/mL). It is noticeable here that the cells in cultivation with P(EtOₓ) consistently achieved higher cell densities than in cultivations with Pluronic® F68. Especially under high shear loads, the cultures with the surfactant POₓ showed significantly increased maximum cell densities, increased viability and a longer culture duration compared to the so far used Pluronic® F68. The viability of the cultures with Pluronic F68 decreased to less than 60% within 3 and 4 days, respectively.

With regard to the concentration range in which P(EtOₓ) was tested here, there were hardly any differences in the maximum live cell density (10.8-12.6-10⁶ cells/mL) in the shaking flasks without baffle. The cultures with increased shear stress showed maximum live cell densities of 3.5-5.6×10⁶ cells/mL, with no direct relation to the concentration used.

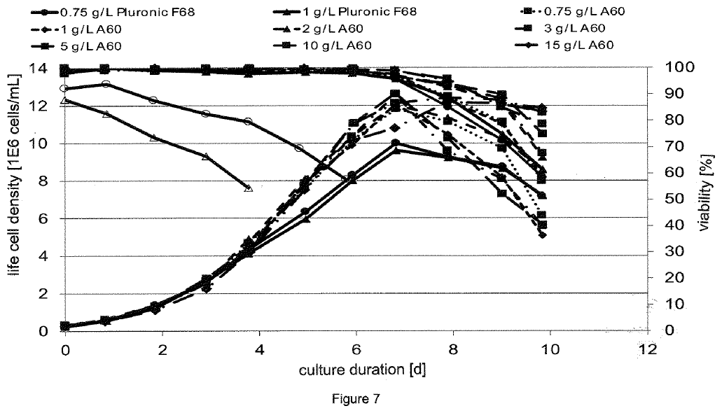

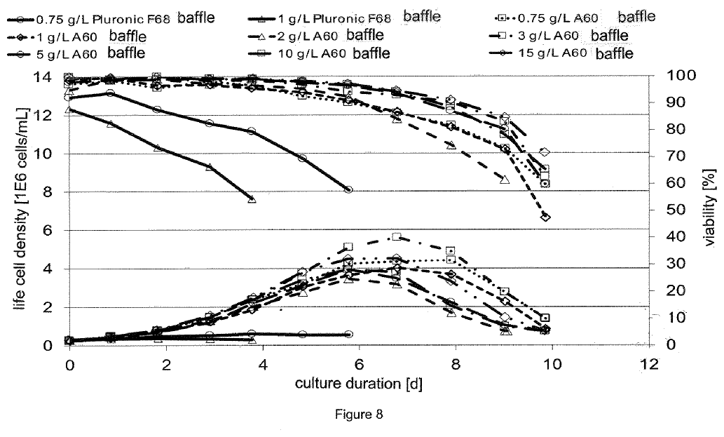

For a better insight, the cultures in FIGS. 7 and 8 are again listed separately by shear stress.

FIG. 7 shows growth curves (solid lines) and development of viability (dashed lines) of the HEK F cells in HEK TF medium in shaking flasks without baffles. Different concentrations Pluronic® F68 (0.75 g/L and 1 g/L) and P(EtOₓ) (0.75-15 g/L) were used as surfactants.

FIG. 8 shows growth curves (solid lines) and development of viability (dashed lines) of the HEK F cells in HEK TF medium in shake flasks with baffles (baffled). Different concentrations Pluronic® F68 (0.75 g/L and 1 g/L) and P(EtOₓ) (0.75-15 g/L) were used as surfactants.

### EXAMPLE 5

### POₓ Lot Testing

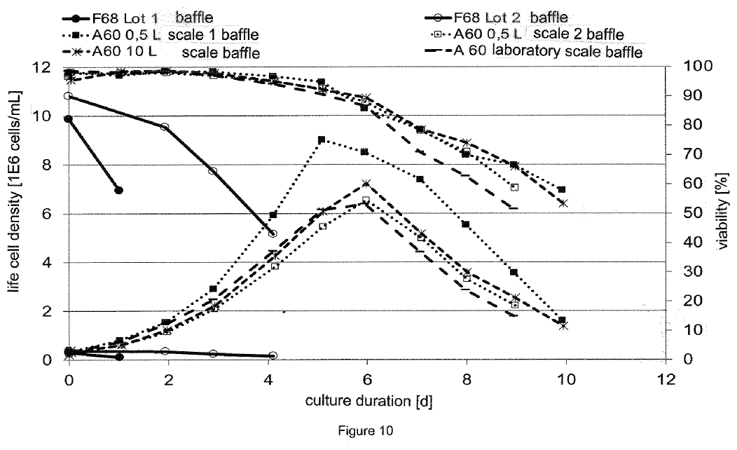

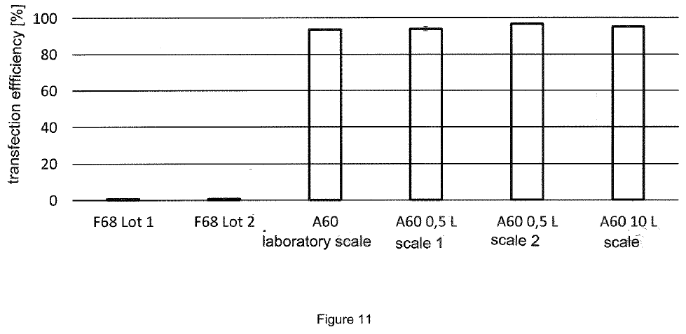

In another preparation, HEK F cells were cultivated in HEK TF medium (serum and protein free, without Pluronic® F68) (order no. 861 (Xell AG, Bielefeld)) with the addition of the various lots of P(EtOₓ) compared to two lots of Pluronic® F68 to identify possible effects of the upscaling of the production process. In order to investigate again the protective property against increased shear forces, this test was carried out both in shaking flasks without baffle and with baffle. The results of this test are shown in FIGS. 9 and 10.

FIG. 9 shows the growth curve and the development of viability of the HEK F cells in HEK TF, adding various lots of P(EtOₓ) from the upscaling of the production process in shake flasks without baffle.

FIG. 9 shows the results in shaking flasks without baffles. With the exception of the Pluronic Lot 1 culture (7.8×10⁶ cells/mL), all cultures showed comparable maximum live cell densities of 9-10, 1×10⁶ cells/mL and no significant differences between the different P(EtOₓ) lots could be detected.

FIG. 10 shows the growth curve and the development of viability of the HEK F cells in HEK TF, adding various lots to P(EtOₓ) from the upscaling of the production process in shake flasks with baffles.

Cultivations in shake flasks with baffles again showed that cells with the addition of P(EtOₓ) have a higher shear tolerance than those with Pluronic® F68 as surfactant; the cultures with Pluronic® F68 showed a decline of viability by 20% (lot 1) and 10% (lot 2) after 24 hours. The cultures with different P(EtOₓ) lots reached maximum live cell densities of 6.4-9×10⁶ cells/mL, with a lot from the 0.5 L production scale showing a higher growth compared to the other lots. Under increased shear stress, only slight differences of the different P(EtOₓ) lots from the upscaling of the production process arose, which were not recognizable in this test under conditions without increased shear stress.

### EXAMPLE 6

### Transfection Efficiency

In order to check the transfection efficiency, two shaking tubes of the cultures from the shaking flasks without baffles were applied and the HEK F cells were transfected with a CFP plasmid by means of polyethyleneimine (PEI). Forty-eight hours after transfection, cell densities, viabilities and the transfection efficiency of these cultures were recorded. The results of these measurements are shown in FIGS. 11 to 13.

FIG. 11 shows mean values of the transfection efficiencies of HEK F cells in HEK TF medium. Two Pluronic® F68 lots and the various lots of the scaling up of P(EtOₓ) production were used as surfactants for cultivation in the shaking tube followed by transfection of a CFP plasmid by means of PEI.

FIG. 11 shows that the transfection efficiencies of the cultures with the different P(EtOₓ) lots were all over 90% (93.5%-96.7%). No significant differences in transfection efficiencies were found between the individual scaling steps. The cultures of the two Pluronic® F68 lots could not be successfully transfected in this experiment, the efficiencies were less than 1%.

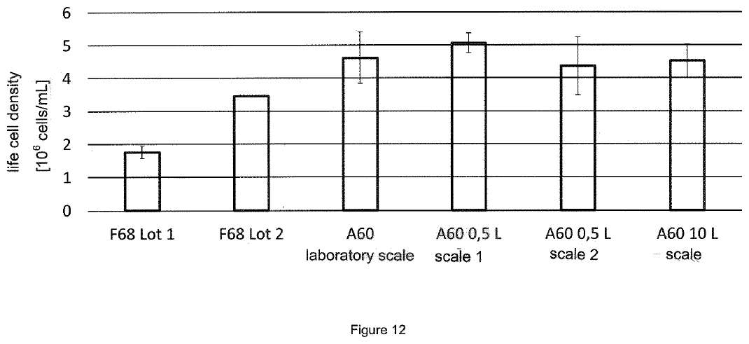

FIG. 12 shows mean values of live cell densities 48 h after transfection of HEK F cells in HEK TF medium. Two Pluronic® F68 lots and the various lots of the scaling up of P(EtOₓ) production were used as surfactants for cultivation in the shaking tube followed by transfection of a CFP plasmid by means of PEI.

FIG. 12 shows the live cell densities 48 h after transfection. Here it can be seen that the mean values of the live cell densities of the cultures with the different P(EtOₓ) lots did not differ significantly. The values varied between 4.4×10⁶ cells/mL and 5.1×10⁶ cells/mL. The two Pluronic® F68 lots had live cell densities of 1.8×10⁶ cells/mL (lot 1) and 3.5×10⁶ cells/mL (lot 2).

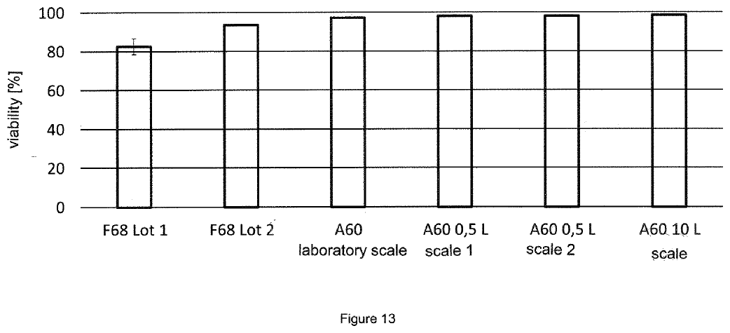

FIG. 13 shows mean values of the viabilities 48 h after transfection of the HEK F cells in HEK TF medium. Two Pluronic® F68 lots as well as the various lots of the scaling up of the P(EtOₓ) production were used as surfactants for the cultivation in the shaking tube with subsequent transfection of a CFP plasmid by means of PEI.

The mean values of the viabilities of the transfected cultures 48 h after transfection can be found in FIG. 13. It can be seen that the PD_{X} cultures showed a high viability between 97.5% and 98.2% and that there was no significant difference between the P(EtOₓ) lots. The viability of the Pluronic® F68 cultures was 82.4% (lot 1) and 93.6% (lot 2).

1. Cell cultures comprising one or more water-soluble poly(oxazolines) in a cell culture medium, provided that the cell culture medium contains essentially no serum.

2. Cell cultures according to claim 1, characterized in that the cell culture medium is essentially free of animal components.

3. Cell cultures according to claim 1, characterized in that the used cell culture medium is serum-free and/or protein-free.

4. Cell cultures according to claim 1, characterized in that the cell culture medium is a chemically defined medium.

5. Cell cultures according to claim 1, characterized in that these contain cells, which are suspended in the cell culture medium.

6. Cell cultures according to claim 1, characterized in that the cells are suspensions-adapted cells.

7. Cell cultures according to claim 1, characterized in that these are used for protein production, virus and/or virus particle production, investigation of metabolism, division and/or other cellular processes.

8. Cell cultures according to claim 1, characterized in that these contain cell lines selected from at least one of the following groups: 293-T, A431, A549, BCP1, bEnd.3, BHK-21, BxPC-3, BY-2, CHO, CMT, COS-1, COS-7, CV-1, EPC, HDMEC-T, HEK, HeLa, HepG2, HL-60, HMEC-1, HUVEC-T, HT-1080, Jurkat, K562, LNCaP, MCF-7, MCF-10A, MDCK II, MDT-1A, MyEnd, Neuro-2A, NIH-3T3-T, NTERA-2 cl.D1, P19, PANC-1, Peer, RTL-W1-T, Sf-9, Saos-2, T2, T84 or U-937.

9. Cell cultures according to claim 8, characterized in that the cell lines are selected from the group consisting of CHO or HEK, in particular HEK 293.

10. Cell cultures according to claim 1, characterized in that these contain hybridoma cells.

11. Cell cultures according to claim 1, characterized in that these contain stem cells.

12. Cell cultures according to claim 1, characterized in that these contain 0.01 to 15 wt. %, preferably 0.075 to 15 wt. %, more preferred 0.05 to 10 wt. %, highly preferred 0.1 to 10 wt. % of water-soluble poly(oxazolines) in relation to the total amount of cell culture.

13. Cell cultures according to claim 1, characterized in that the water-soluble poly(oxazoline) contains at least 80 wt. %, based on its total mass, of repeat structural units of formula I and/or formula II
(I), —NR¹—CH₂—CH₂—CH₂— (II),
in which
R¹ means a residue of formula —CO—R²,
R² is selected from the group consisting of hydrogen, methyl, ethyl, —CₘH₂ₘ—X or —(CₙH₂ₙ—O)ₒ-(CₚH₂ₚ—O)_{q}—R³,
R³ is hydrogen or C₁—C₆-alkyl,
m is an integer from 1 to 6,
X is selected from the group consisting of hydroxyl, alkoxy, amino, N-alkylamino,
N,N-dialkylamino, carboxyl, carboxylic ester, sulfonyl, sulfonic acid ester or carbamate,
n and p independently of one another are integers from 2 to 4, wherein n is unlike p, and
o and q independently of one another are integers from 0 to 60, wherein at least one of o or q is unlike 0.

14. Cell cultures according to claim 13, characterized in that the water-soluble poly(oxazoline) has at least 90 wt.-%, based on its total mass, of repeat structural units of formula I, wherein R² means methyl or ethyl.

15. Cell cultures according claim 1, characterized in that these contain one or more active ingredients, in particular pharmaceutically active ingredients and/or vaccines.

16. A method for protein production, virus and/or viral particle production, investigation of metabolism, division and/or other cellular processes comprising the cultivation of cell cultures comprising one or more water-soluble poly(oxazolines) in a cell culture medium, provided that the cell culture medium contains essentially no serum.

17. The method according to claim 16, characterised in that it also contains the following measures:
(i) filling a bioreactor with one of the cell cultures, and
(ii) agitating the contents of the bioreactor.

18. The method according to claim 16, characterized in that the duration of the cultivation of the cells in the case of a batchwise method is 5 hours to 30 days, preferably 5 hours to 21 days and in particular 10 hours to 15 days and in the case of a continuous process is 10 days to 180 days, preferably 10 days to 60 days and in particular 15 days to 35 days.

19. The method according to claim 17, characterized in that the content of the bioreactor is stirred.

20. The method according to claim 17, characterized in that the bioreactor contains baffles for the generation of turbulence.

21. (canceled)

22. (canceled)

23. (canceled)

24. (canceled)

25. (canceled)
